(19) United States
(12) Patent Application Publication
(10) Pub. No.: US 2015/0216595 A1
(43) Pub. Date: Aug. 6, 2015

(54) OPTIMIZED METHOD OF COATING THE MICROWAVE ABLATION PROBE FOR SURGICAL APPLICATION

(71) Applicant: Microsurgeon, Inc., West Hollywood, CA (US)

(72) Inventors: Babak Kateb, Brentwood, CA (US); Edward Dean Hammerslag, Thousand Oaks, CA (US); Hosmel Galan, Camarillo, CA (US)

(73) Assignee: Cedars-Sinai Medical Center, Los Angeles, CA (US)

(21) Appl. No.: 14/614,741

(22) Filed: Feb. 5, 2015

Related U.S. Application Data
(60) Provisional application No. 61/936,786, filed on Feb. 6, 2014.

(51) Int. Cl. A61B 18/18

(52) U.S. Cl. CPC A61B 18/1815; A61B 2018/00446

(57) ABSTRACT

An ablation probe which includes a probe body with a distal and proximal end, an ablation tip at the distal end of the probe body, an anode provided proximate to the ablation tip, a coaxial cable disposed within the probe body and coupled to the ablation tip and anode so that electromagnetic energy of a predetermined frequency is communicated thereto for dielectric heating of tissue, and a Parylene C coating on at least the ablation tip and anode to electrically isolate the ablation tip and anode from tissue without interference with microwave energy transmission through the probe to the tissue. The Parylene C is a vapor deposited coating on at least the ablation tip and anode to electrically isolate the ablation tip and anode from tissue.

### RELATED APPLICATIONS

The present application is related and claims priority to U.S. Provisional Application Ser. No. 61/936,786, filed on Feb. 6, 2014, pursuant to 35 USC 119, which application is incorporated herein in its entirety.

### FIELD OF THE INVENTION

The disclosure relates to the field of surgical microwave ablation probes to treat cancers or any disorders of brain, breast, liver, pancreas/GI, prostate and head and neck.

### BACKGROUND

Ablative procedures increase the tumor temperature (50° C. to 80° C.), which modifies tumor cell morphology and function. The heat causes coagulation of cellular proteins and leads to tumor cell death when temperatures exceed 50° C. (Nikfarjam et al. 2005). Microwave ablation is a newer technology that utilizes an electromagnetic field to vibrate the water molecules in the treated tissue. This generates the heat that causes disruption of cellular proteins and apoptosis (cell death). This method has certain advantages over RF ablation as described below.

Radiofrequency ablation (RFA) is a common and well-known ablation technique used to ablate brain tissue in diseases such as Parkinson's disease and also brain tumors (Anzai et al. 1995). This technology is based on the conduction of alternating current up to 500 kHz through the tissue, which produces ionic agitation of the cells due to tissue resistance to the alternating current. This maximizes the current around the probe and enables the device to concentrate the heat around the probe and generate an oval lesion. While this technique is effective, it has its flaws. The limitations of RFA relate to its physics.

In this technique, current flows unilaterally away from the electrode, creating a “time-average power deposition decay rate”. The formula to calculate such power loss is: P≈1/r4, where P=power and r=radius. This limits the volume of tumor that can be treated, as it becomes more difficult to conduct heat the further the treatment area is from the probe. As a result, RFA probes cause tissue charring at the center of the lesion. Lesion volume also correlates poorly with RF dose (Anzai 1997). The heat generated is not uniformly distributed, which may enable cancer cells to grow back within the lesioned area. Potential for incomplete tumor ablation is also reported due to a heat sink effect, in which blood vessels absorb heat away from the surrounding tissue. Additionally, RFA requires a ground skin patch and several cases of skin burn have been reported (Martin et al. 2010). Therefore, there is a need to develop technology without these limitations. One such technology is microwave ablation, which creates heat by vibrating dipole water molecules.

Microwave (MW) covers the electromagnetic spectrum between frequencies of 300 MHz to 300 GHz with wavelengths around 1 m to 1 mm. RFA and microwave ablation (MWA) are somewhat similar techniques; however microwave ablation is more effective in creating larger lesions at a greater depth and within a shorter time period (within 5 minutes). MWA lesion volumes are proportional in size to the duration of energy and power, as an increase in MW power results in significant increase in lesion volume without charring. Furthermore, MWA devices do not require a skin ground patch and have the ability to generate large lesions in the presence of blood perfusion without creating a heat sink effect.

Microwave ablation has been safely used in the treatment of cancers of the liver, kidney, prostate, and lung in addition to treatment of cardiac disease (Knavel et al. 2013). Microwave balloon angioplasty (MBA) is utilized to enlarge the lumen of narrowed cardiac arteries, which may prevent restenosis. Dysfunctional uterine bleeding has also been treated using microwave technology (Nakayama et al. 2013). Thus, the body of literature suggests that MWA could be safely used in the treatment of brain tumors.

There are approximately 30,000 cases of newly diagnosed primary malignant human brain tumors in the United States each year. The standard treatments for primary malignant tumors consist of attempts at maximum surgical resection followed by radiation and chemotherapy. Malignant brain tumors are often fatal within one to two years of diagnosis despite multimodality treatment. Although tumor resection improves patient survival, open surgical resection carries significant risk and is not possible in approximately 30% of cases. This is due to clinical factors such as patient age, comorbidities, and tumor characteristics such as tumor location, size and infiltration. Furthermore, the percentage of inoperable tumors rises with tumor recurrence. Given that greater extent of resection is strongly associated with longer survival in malignant gliomas, new minimally invasive treatments that have the potential to ablate tumor tissue are being explored. Ablation may have the same biological effect as surgical resection and hence, has the potential of improving outcomes in patients; especially of those patients that are ineligible for open surgical resection. In this pilot study, we are assessing the safety of microwave thermal ablation. If deemed safe by initial studies, this technique has the potential to treat a significant proportion of patients with malignant brain tumors. Given the strong association of extent of resection and survival, patients with malignant brain tumors who are not candidates for open surgical resection or who have failed radiation therapy may benefit significantly from this ablation technology in the future.

### BRIEF SUMMARY

A neurological microwave ablation probe is used to mediate brain tissue by insertion through a burr hole in the skull and application of the probe into a brain tumor to be microwave heated and killed. The heating of the tissue is intended to occur from noncontact dielectric heating as opposed to ohmic heating by direct conduction of microwave current in the tissue, which could result in uncontrolled current concentrations and local tissue burning instead of regional or uniform heating. While such neurological microwave ablation probes are conventionally insulated with one or more layers of insulation to prevent direct electrical connection of the microwave current to the tissue, it is the object of the invention to provide electrical isolation of the probe tip from the tissue to an extent more complete and more secure than achieved by conventional electrical insulation.

According to the invention it has been determined that improved microwave ablation probe tip isolation is achieved if the probe tip and anode is coated with elastomeric polymer, or more specifically parylene C. Parylene C is characterized by one chlorine group per repeat unit on the main-chain phenyl ring. Because of its higher molecular weight parylene C has a higher threshold temperature, 90° C., and therefore has a much higher deposition rate, while still possessing a high degree of conformality. It can be deposited at room temperature while still possessing a high degree of conformality and uniformity and a moderate deposition rate>1 nm/s in a batch process. As a moisture diffusion barrier, the efficacy of a coating scales non-linearly with their density. Halogen atoms such as F, Cl and Br add density to the coating and therefore allow the coating to be a better diffusion barrier.

In conventional construction it is possible that in some cases fluid leakage under the discrete insulation layers provided to cover the ablation tip could occur from time to time or that microscopic cracks or other defects in the insulation layer could allow electrical conduction therethrough, particularly through conductive intermediate fluids occurring in the surgical theater. The entire microwave antenna or cathode/anode structure is completely coated so that no contact directly or through intermediate fluids is possible between the microwave or microwave active elements and the brain tissue. The parylene C coating is not prone to cracking or electrical leakage, but similarly does not interfere with or alter the electromagnetic transmission of energy therethrough at the frequency of use, namely 2.45 GHz.

The problem to solve is to have a biocompatible seal over the probe materials that is adequately robust to remain electrically and mechanically uncompromised throughout its life up to and including the surgical procedure. At the same time the material needs to provide minimal interruption of the microwave energy field that provides the thermal treatment of the tissue. Parylene is an insulator to direct current but can be applied at such a small thickness as to provide an adequately low capacitive reactance to the microwave path to have insignificant effect on the field strength and path.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The disclosure can be better visualized by turning now to the following drawings wherein like elements are referenced by like numerals.

### BRIEF DESCRIPTION OF THE DRAWINGS

The specification contains at least one drawing executed in color. Copies of this patent or patent application publication with color drawing(s) will be provided by the Office upon request and payment of the necessary fee.

FIG. 1 is a photograph of the distal end of the microwave ablation probe at a beginning step of its manufacture.

FIG. 2 is a photograph of the distal end of the microwave ablation probe after the installation of the pair of thermistors into slots defined in the fiberglass core rod.

FIG. 3 is a photograph of the distal end of the microwave ablation probe showing the folding back of the thermistors and temporarily taping them to the core rod.

FIG. 4 is a photograph of the proximal end of the microwave ablation probe, folding of the thermistor wires and tensioning them in the core rod and corresponding slots.

FIG. 5 is a photograph of the distal end of the microwave ablation probe after the insertion of the microcoaxial cable through the rod core and extending from the distal tip.

FIG. 6 is a photograph of the distal end of the microwave ablation probe after soldering on the ablation tip to the center conductor of the coax cable.

FIG. 7 is a photograph of the distal end of the microwave ablation probe after drawing the ablation tip up snug against the distal end of the rod core and affixation thereto.

FIG. 8 is a photograph of the distal end of the microwave ablation probe after removal of the tape and arrangement of the thermistor wires in preparation to drawing them snugly into the corresponding slots in the rod.

FIG. 9 is a photograph of the distal end of the microwave ablation probe after the thermistors have been nested into their corresponding slots and affixed therein.

FIG. 10 is a photograph of the distal end of the microwave ablation probe in enlarged view illustrating the potting of the thermistors into their slots.

FIG. 11 is a photograph of the distal end of the microwave ablation probe after the filling in of the anode bore with conductive epoxy.

FIG. 12 is a photograph of the distal end of the microwave ablation probe after coating with parylene and the installation of a heat shrink tubing over the distal portion thereof.

FIG. 13 is a photograph of the proximal end of the microwave ablation probe after affixation to the handle assembly.

FIG. 14 is a photograph of the proximal end of the microwave ablation probe after wrapping of the thermistor wires with electrical tape.

FIG. 15 is a photograph of the proximal end of the microwave ablation probe showing the installation of the elastomeric handle.

FIG. 16 is an enlarged photograph of the proximal end of the microwave ablation probe and affixation of the elastomeric handle to the probe.

FIG. 17 is a photograph of the proximal end of the microwave ablation probe showing insertion of the partially constructed probe into a handle half.

FIG. 18 is a photograph of the proximal end of the microwave ablation probe showing both handle halves assembled and affixed together while being held in a clamp during curing of the adhesive.

The disclosure and its various embodiments can now be better understood by turning to the following detailed description of the preferred embodiments which are presented as illustrated examples of the embodiments defined in the claims. It is expressly understood that the embodiments as defined by the claims may be broader than the illustrated embodiments described below.

### DETAILED DESCRIPTION OF THE PREFERRED EMBODIMENTS

One ablation system in which the probe could be used is substantially similar to that disclosed in U.S. Pat. No. 5,301,687 and U.S. Pat. No. 6,706,040, incorporated herein by reference.

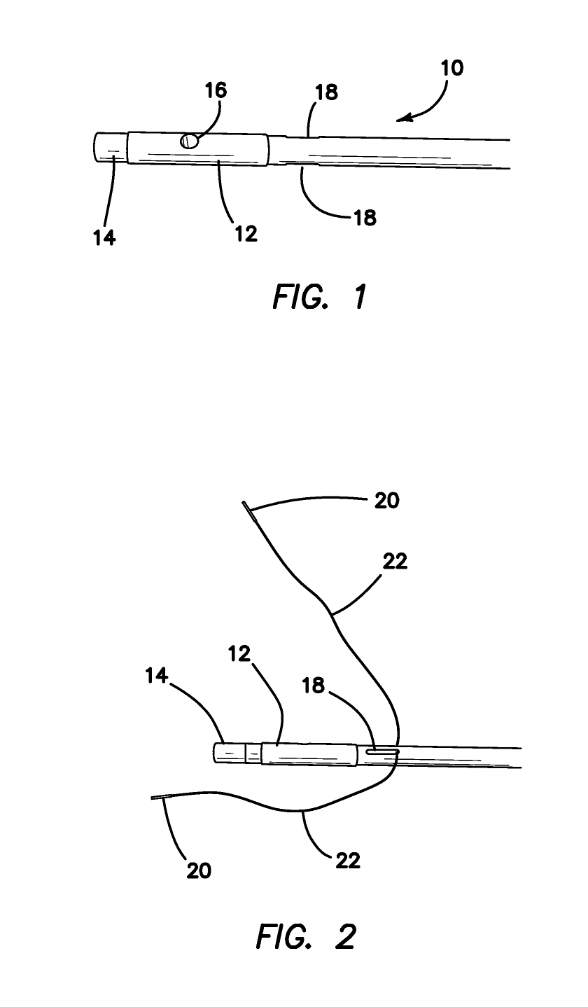

FIG. 1 is a diagram showing the first step in the manufacture of the microwave ablation probe 10 of the invention, wherein the distal end of ablation probe 10. The probe 10 includes a hollow fiberglass tube 14 having an outer diameter of 2.3 mm and an inner diameter of 1.6 mm, serving as a core of the probe 10. In the illustrated embodiment probe 10 is 7.5 inch (19.05 mm) long. A metallic anode sleeve 12 made of Brass 260 Alloy is slid over the distal end of tube 14 and spaced 0.075±0.010 inch (1.905±0.25 mm) from the end. A 0.03 inch (0.762 mm) through-bore hole 16 is drilled or defined completely through anode 12 and tube 14. A pair of diametrically opposing slots 18 are defined through the walls of tube 14 extending from the proximal end of anode 12 for approximately 0.14 inch (3.56 mm). Bore 16 has an axis perpendicular to the longitudinal axis of tube 14. Slots 18 lie in a plane perpendicular to the axis of bore 16.

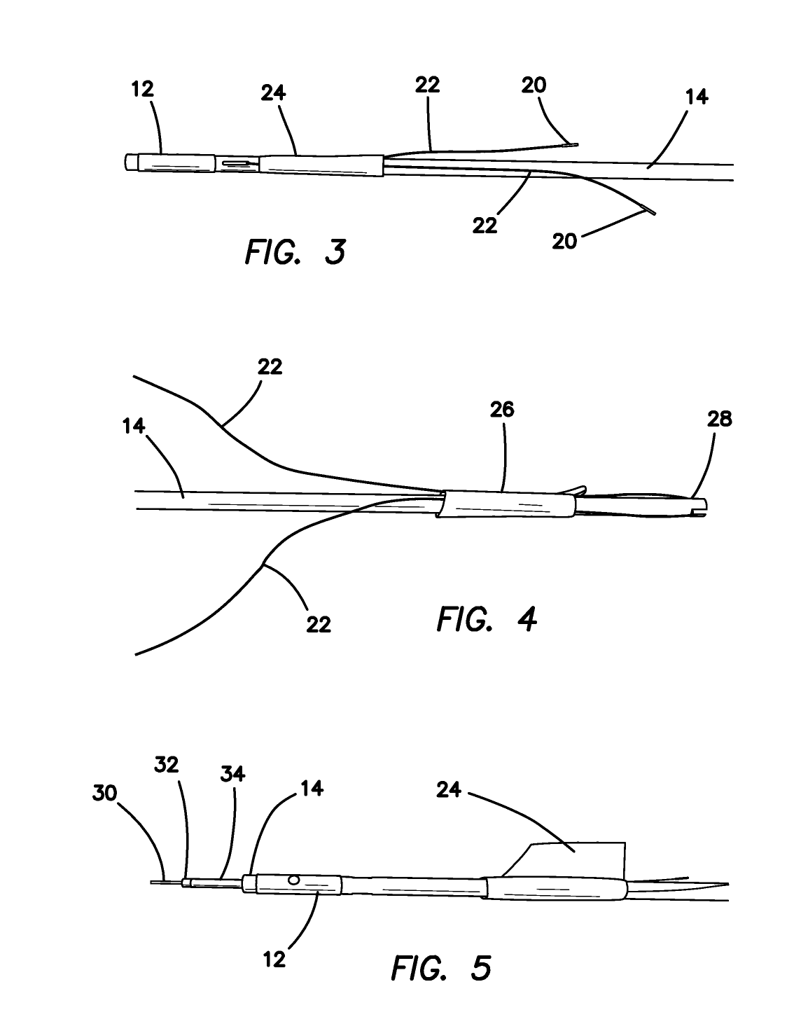

In the next step two conventional thermistors 20 with wire leads 22 are led through tube 14 out through each one of the two slots 18 as shown in FIG. 2. Leads 22 of thermistors 20 are folded back along the length of tube 14 as shown in FIG. 3 and temporarily taped to tube 14 by a wrap of tape 24. The opposing ends of leads 22 of thermistors 20 are pulled taut through the proximal end 28 of tube 14 and then temporarily taped into place by a wrap of tape 26 as shown in FIG. 4. In this manner leads 22 extending through slots 18 are kept snugly retained in slots 18 against the inner and outer wall of tube 14.

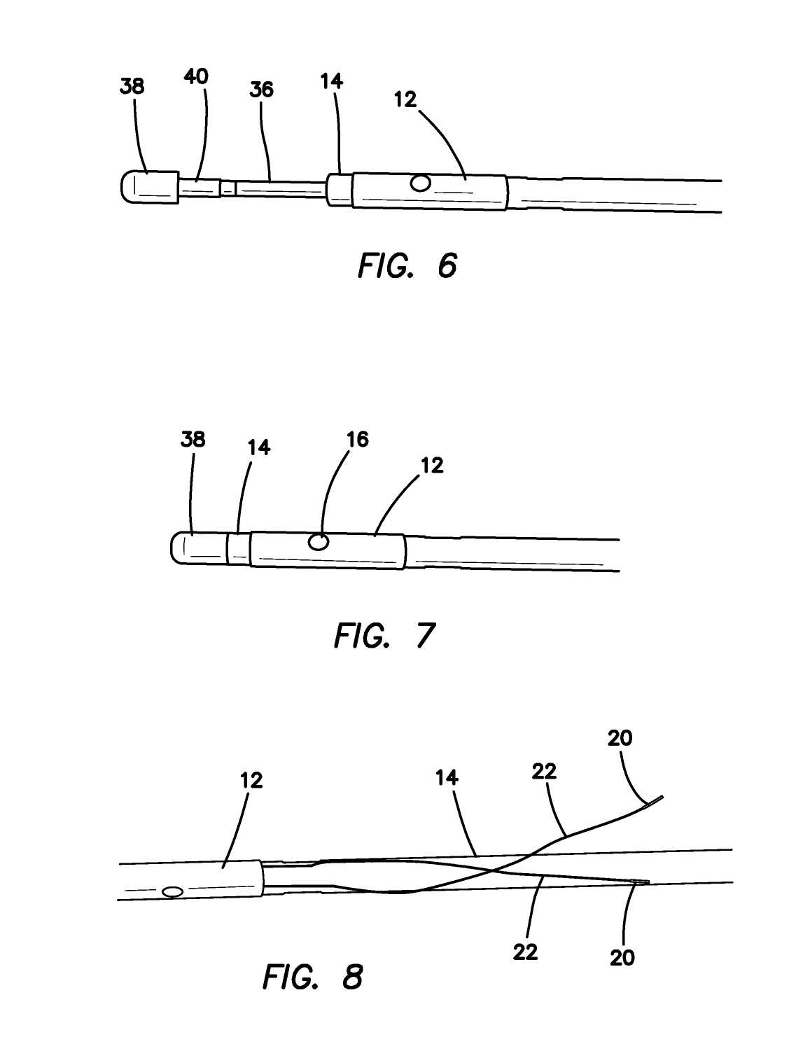

In the next step a length of semi-rigid microcoaxial cable with the center conductor 30 bared and the center core 32, shielding 34 removed from a distal segment is disposed through tube 14 and extended from its distal end as seen in FIG. 5. A probe tip 38 is then soldered onto the center conductor 30 of the microcoaxial cable as depicted in FIG. 6. The probe tip 30 is then drawn flush to the distal end of tube 14 by pulling back on the microcoaxial cable from the proximal end of tube 14. In the illustrated embodiment, probe tip has a reduced diameter proximal portion 40, which is coated with adhesive and sized to be snugly drawn into the distal end of tube 14 where it is fixed as shown in FIG. 7.

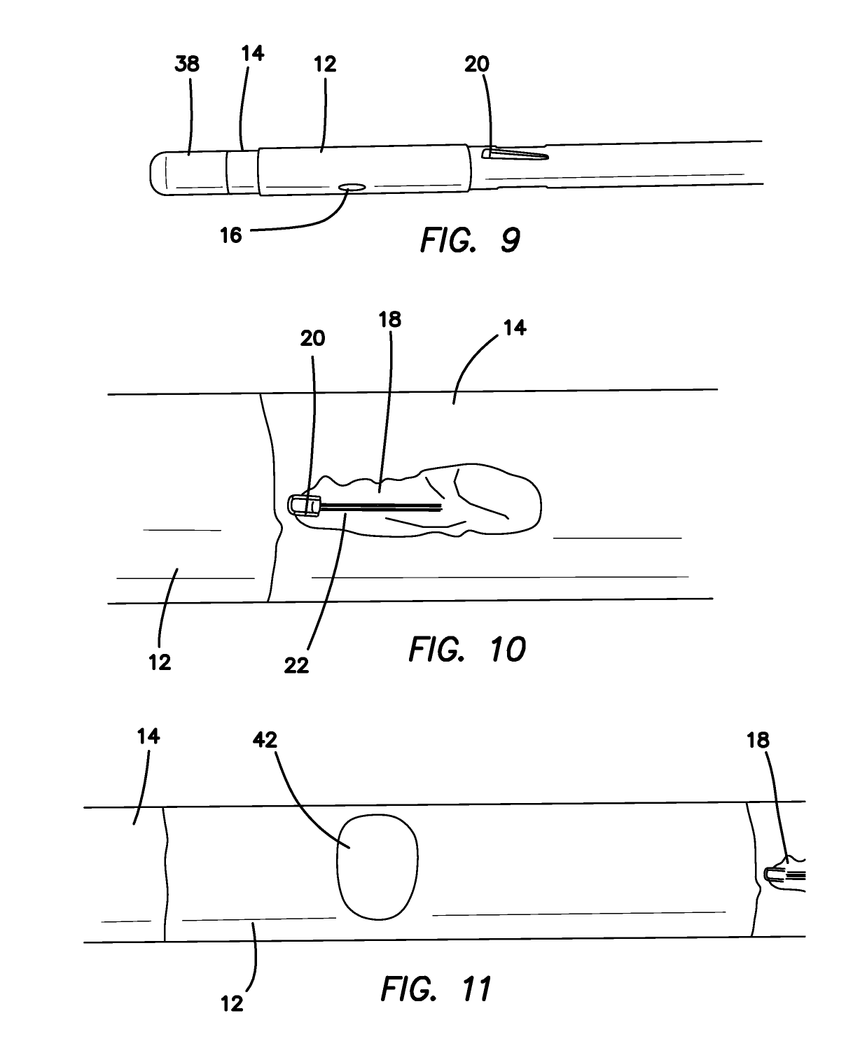

Tape wraps 24 and 26 are removed from tube 14 as shown in FIG. 8 freeing thermister leads 22. Leads 22 are straightened and then withdrawn into tube 14 until thermistors 20 are each snugly seated in their corresponding slots as shown in FIG. 9. The integrity of the thermistors 20 are electrically checked to insure that there has been no damage to them during assembly and they are microscopically checked as seen in the magnified view of FIG. 10 to insure that there are no cracks or damage to the glass bead that forms the head of thermistor 20. Thermistors 20 are then potted with epoxy within their corresponding slots 18. A conductive epoxy 42, such as silver epoxy, is prepared and used to fill in bore 16 in anode 12 to make an electrical connection with shielding 34 of the microcoaxial cable disposed through tube 14 and underlying bore 16. Electrical continuity between shielding 34 at the proximal end of tube 14 and anode 12 is then checked.

Probe 10, prepared as disclosed above, is now ready for coating with parylene C supplied by Para Tech of Aliso Viejo, Cali. The parylene C is coated onto the entire length of probe 10 or at least onto its distal portion include probe tip 38 and anode 12 by Para Tech. The problem which needed to be solved is to have a biocompatible seal over the probe elements that is adequately robust to remain electrically and mechanically uncompromised throughout its life up to and including the surgical procedure. The thickness and the manner in which this cover or coating is put on to the probe could block or alter the radiation characteristics of the microwaves transmitted from anode 12 and probe tip 30, if the process is not done properly. The coating must minimally interrupt the microwave transmitted energy, that is the source of the dielectric heating of the tissue and hence the thermal treatment of the tissue. Parylene C is an insulator to direct current but can be applied at such a small, controlled thickness so that a low microwave capacitive reactance is presented to the microwave path and there is an insignificant effect on the field strength and path. The disclosed approach allows the entire probe to be covered without any interference with microwave emission thereby making the entire device functional.

The Parylene C coating is applied to the probe 10 with a vacuum deposition process. The Parylene C coating process used at Para Tech is superior for controlling temperature and pressure during the coating cycle and is unmatched in coating quality as well as precise production control. The coating cycle begins with vaporization of the powdered raw material (dimer) at 150° C., creating a dimeric gas. Gas molecules are subsequently cleaved to the monomer form in a second stage by heating to 650° C. The active monomer gas is then introduced to an evacuated coating chamber where it disperses and polymerizes spontaneously on substrate surfaces at room temperature to form Parylene C film. Unlike a curing liquid coating, this molecular stage activity produces no stress or surface tension on coated surfaces. The monomer gas disperses evenly throughout the chamber. It exhibits no liquid properties such as surface tension or meniscus, and that all sides of every surface are exposed simultaneously to the polymerizing gas, including flat surfaces, sharp edges, slots and crevices.

The coating is approximately 2-3 microns thick. Integrity of the coating is confirmed by checking for the lack of electrical continuity between probe tip 38 and the center conductor 30 of the microcoaxial cable extending from the proximal end of tube 14. Thus the coating is thick enough to provide adequate electrical insulation for the RF current and voltage applied to the probe to prevent any direct electrical conduction into the tissue, but is thick enough not to interfere with or to materially attenuate with the RF radiation of energy from the probe into the tissue. Thermistors 20 are again are visually checked with a magnified viewer or microscope for cracks or other damage.

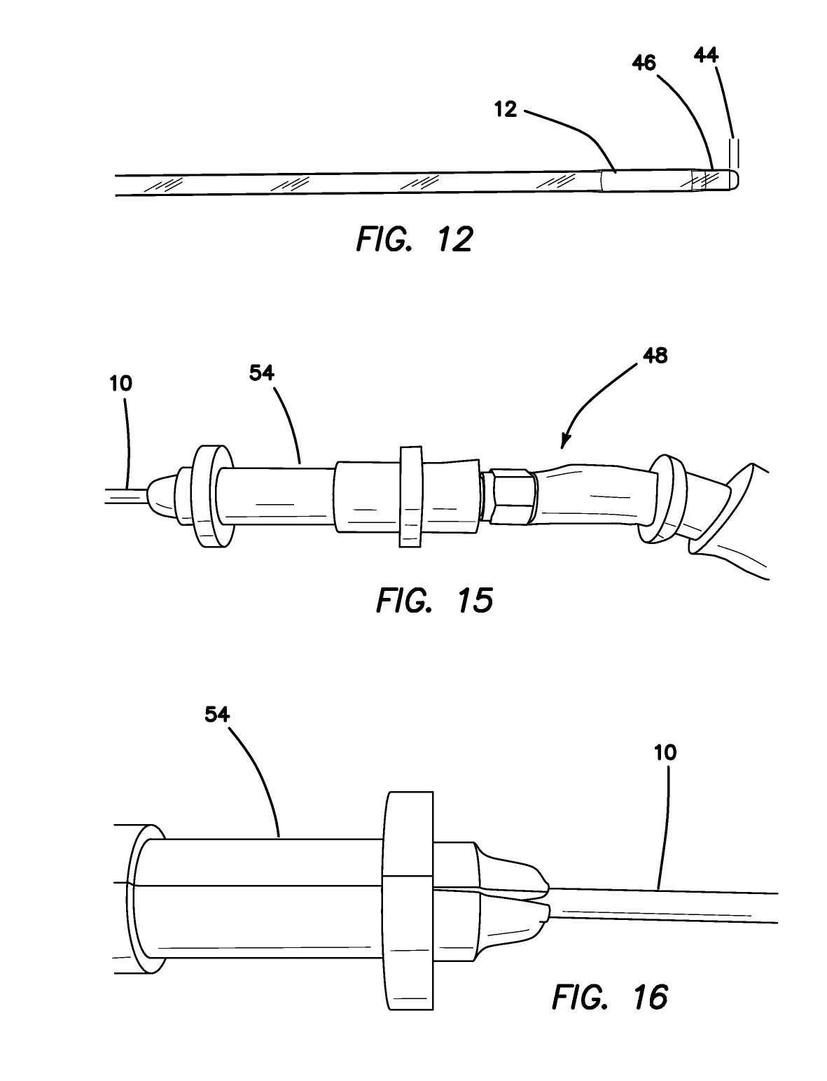

A length of 6-6.25 inches (152.4-158.75 mm) of surgical grade Teflon® FEP heat shrink tubing 46 is cut and telescopically disposed over the distal end of probe 10 as shown in FIG. 12. The end of tubing 46 is positioned to extend between 0.00 to 0.06 inch (0.0-1.52 mm) from the distal end of probe tip 38 to provide an end spacing 44. The end spacing 44 is maintained while the heat shrink tubing is snugly contracted onto probe 10 by heating from the distal tip of probe 10 toward its proximal end.

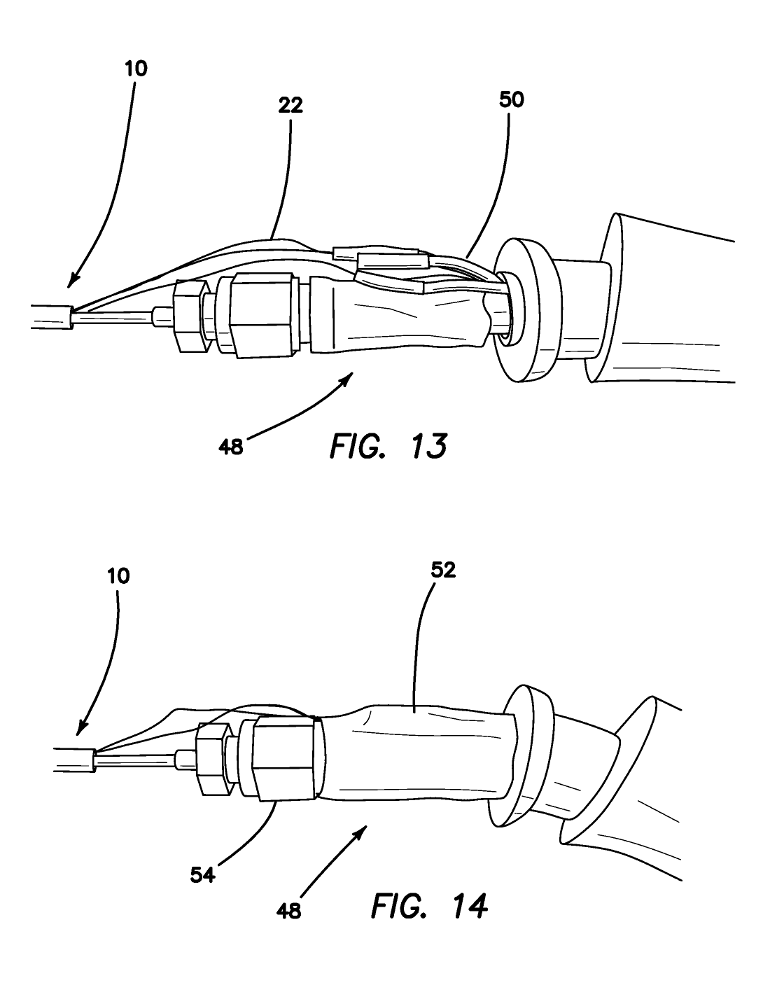

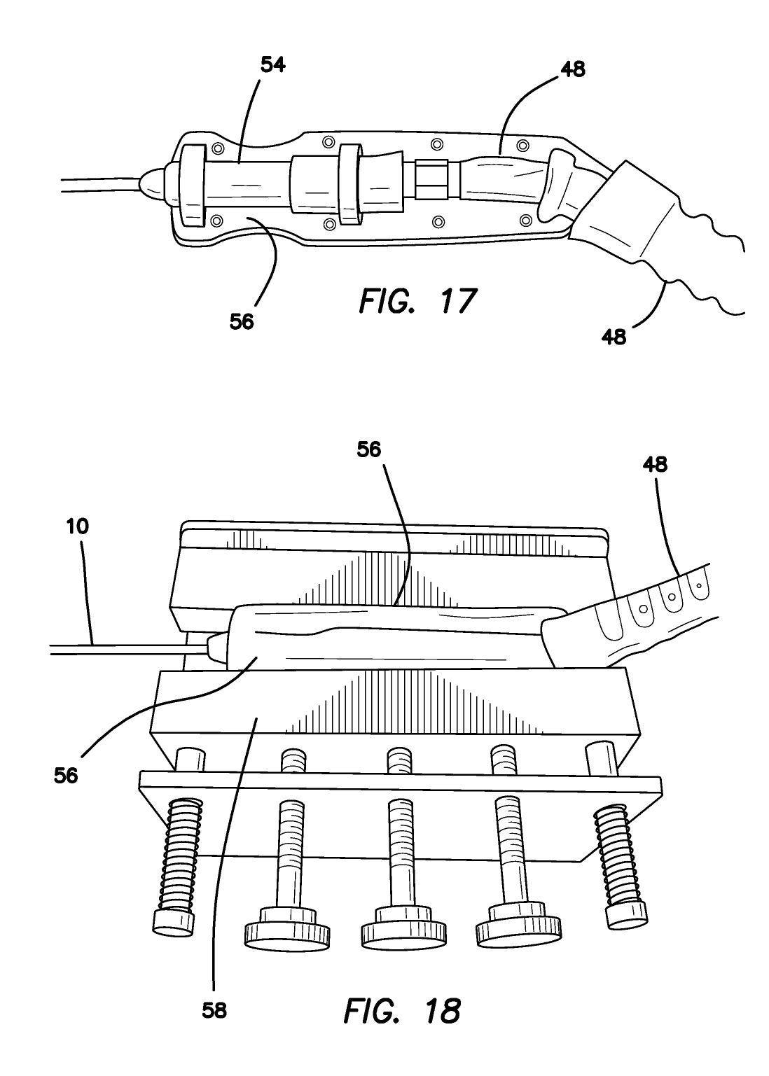

Probe 10 then onto cable assembly 48 as shown in FIG. 13 connecting to the microcoaxial cable. Leads 22 are each provided with heat shrink tubing, trimmed, stripped and soldered to cable assembly 50. The solder joints are wrapped with electrical tape 52 shown in FIG. 14. Note that in FIGS. 13 and 14 a flat of the hex head of the connector on assembly 48 is facing downward in the figures. A two-part elastomer block 54 is then assembled over the distal portion of probe 10 as shown in FIG. 15 with the proximal end of block 54 flush against assembly 48. Adhesive to inserted in any gap between the halves of the block 54 to keep it integral and clamped onto probe 10 as shown in FIG. 16, particularly at its distal end. The assembled probe 10 is then inserted into one half of the handle assembly 56 as shown in FIG. 17. A conforming cavity is defined in handle assembly 56 to allow for a snug conforming fit of the assembled probe 10 therein. Adhesive to added to post and hole locations on the handle assembly half 56 and it is mated with a conforming mating handle assembly half shown in FIG. 18. FIG. 18 shows the two handle assembly halves 56 being clamped together in a jig 58 until the adhesive is set. Electrical integrity of the probe 10 is once again is checked to insure that there has been no damage and the assembly of the microwave ablation probe 10 is complete and ready for labeling.

Included in the final electrical check is a measurement of the resistance of the installed thermistors. In the illustrated embodiment a DC resistance of 18-29 kΩ is regarded as acceptable with a visual inspection for cracks in the thermistor beads 20. Electrical continuity between the anode 12 and hex nut of handle assembly 48 of 10Ω is deemed acceptable. The integrity of the parylene coating is measured by checking the DC resistance from the coated distal end of ablation tip 38 to the center conductor at the proximal end of the microcoaxial cable of greater than 10 MΩ.

Many alterations and modifications may be made by those having ordinary skill in the art without departing from the spirit and scope of the embodiments. Therefore, it must be understood that the illustrated embodiment has been set forth only for the purposes of example and that it should not be taken as limiting the embodiments as defined by the following embodiments and its various embodiments.

Therefore, it must be understood that the illustrated embodiment has been set forth only for the purposes of example and that it should not be taken as limiting the embodiments as defined by the following claims. For example, notwithstanding the fact that the elements of a claim are set forth below in a certain combination, it must be expressly understood that the embodiments includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the embodiments is explicitly contemplated as within the scope of the embodiments.

The words used in this specification to describe the various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the embodiments.

We claim:

1. An ablation probe for dielectric heating of tissue comprising:
a probe body with a distal and proximal end;
an ablation tip at the distal end of the probe body;
an anode provided proximate to the ablation tip;
a coaxial cable disposed within the probe body and coupled to the ablation tip and anode so that electromagnetic energy of a predetermined frequency is communicated thereto for the dielectric heating of tissue; and
a parylene C coating on at least the ablation tip and anode to electrically isolate the ablation tip and anode from tissue without interference with microwave energy transmission through the probe into the tissue for dielectric heating.

2. The ablation probe of claim 1 where the Parylene C coating comprises a diffusion barrier.

3. The ablation probe of claim 1 used to dielectrically heat brain tissue and where the ablation tip and anode act as a microwave antenna and wherein the Parylene C coating completely coats the ablation tip and anode so that no contact directly or through intermediate fluids is possible between the microwave antenna and the brain tissue.

4. The ablation probe of claim 1 where the predetermined frequency is 2.45 GHz and where the Parylene C coating does not materially alter coupling of the radiation of the 2.45 GHz electromagnetic energy from the ablation tip and anode into the tissue.

5. The ablation probe of claim 1 where the Parylene C coating is vacuum deposited on the ablation tip and anode.

6. The ablation probe of claim 1 where the Parylene C coating is 2-3 microns thick on the ablation tip and anode.

7. The ablation probe of claim 1 where the Parylene C coating isolates the ablation tip and anode from any contact with any fluid during use.

8. An optimization in an ablation probe having an ablation tip and anode for microwave dielectric heating of tissue comprising a Parylene C vapor deposited coating on at least the ablation tip and anode to electrically isolate the ablation tip and anode from tissue.

9. The improvement of claim 8 further comprising a probe body with a distal and proximal end.

10. The improvement of claim 9 further comprising a coaxial cable disposed within the probe body and coupled to the ablation tip and anode so that electromagnetic energy of a predetermined frequency is communicated thereto for the dielectric heating of tissue.

11. The improvement of claim 10 further comprising a source of microwave energy coupled to the coaxial cable.

12. The ablation probe of claim 8 used to dielectrically heat brain tissue and where the ablation tip and anode act as a microwave antenna and wherein the Parylene C coating completely coats the ablation tip and anode so that no contact directly or through intermediate fluids is possible between the microwave antenna and the brain tissue.

13. The ablation probe of claim 8 where the predetermined frequency is 2.45 GHz and where the Parylene C coating does not materially alter coupling of the radiation of the 2.45 GHz electromagnetic energy from the ablation tip and anode into the tissue.

14. The ablation probe of claim 8 where the Parylene C coating is vacuum deposited on the ablation tip and anode.

15. The ablation probe of claim 8 where the Parylene C coating is 2-3 microns thick on the ablation tip and anode.

16. The ablation probe of claim 8 where the Parylene C coating isolates the ablation tip and anode from any contact with any fluid during use.

17. An optimized method of coating an ablation tip and anode of a probe used to dielectrically heat tissue with microwaves comprising applying a Parylene C coating to completely cover the ablation tip and anode using a vacuum deposition process by: vaporizing a powdered raw material (dimer) at 150° C. to create a dimeric gas; cleaving the molecules of the dimeric gas to a monomer form by heating to 650° C.; and introducing the active monomer gas to an evacuated coating chamber holding the probe having the ablation tip and anode, and where the monomer gas disperses and polymerizes spontaneously on surfaces of the ablation tip and anode at room temperature to form a Parylene C coating, so that no stress or surface tension is created on coated surfaces where all sides of every surface are exposed simultaneously to the polymerizing gas, including flat surfaces, sharp edges, slots and crevices.

18. The method of claim 17 where introducing the active monomer gas to an evacuated coating chamber forms the Parylene C coating with a thickness of 2-3 microns on the ablation tip and anode.

19. The method of claim 17 where introducing the active monomer gas to an evacuated coating chamber forms the Parylene C coating which isolates the ablation tip and anode from any contact with any fluid during use.

20. The method of claim 17 where introducing the active monomer gas to an evacuated coating chamber forms the Parylene C coating which does not materially alter coupling of 2.45 GHz electromagnetic energy from the ablation tip and anode of the probe into the tissue.
